(19) United States
(12) Patent Application Publication
(10) Pub. No.: US 2007/0254317 A1
(43) Pub. Date: Nov. 1, 2007

(54) Method for Detecting the Activatable Free Form of Psa and the Use Thereof for Diagnosing Benign Pathologies of the Prostate and Adenocarcinoma of the Prostate

(75) Inventors: Sandrine Busseret-Michel, Lyon (FR); Colette Jolivet-Reynaud, Saint Bonnet De Mure (FR)

Correspondence Address: OLIFF & BERRIDGE, PLC, P.O. BOX 19928, ALEXANDRIA, VA, 22320, US

(73) Assignee: bioMERIEUX, MARCY L'ETOILE (FR)

(21) Appl. No.: 10/590,272

(22) PCT Filed: Mar. 1, 2005
(86) PCT No.: PCT/FR05/50132

§ 371 Date: Aug. 22, 2006

(30) Foreign Application Priority Data
Mar. 3, 2004 (FR) 0450426

(51) Int. Cl. G01N 33/53

(52) U.S. Cl. 435/7.23

(57) ABSTRACT

The present invention relates to a method for the in vitro diagnosing of a benign pathology of the prostate or of an adenocarcinoma of the prostate, characterized in that it comprises the step consisting of detection, in a biological sample from a patient suspected of suffering from a benign pathology of the prostate or from an adenocarcinoma of the prostate, of the activatable free form of PSA. This method of diagnosis can be carried out by means of the steps consisting in: i) bringing a binding partner capable of binding specifically to activatable free PSA into contact with a biological sample from a patient suspected of suffering from a benign pathology of the prostate or of an adenocarcinoma of the prostate, ii) demonstrating the capture of the activatable free form of PSA by said binding partner, iii) calculating the ratio of the amount of activatable free form of PSA detected in step ii) to the amount of a form of PSA other than the activatable free form, present in a sample of the same nature taken from the same individual, and iv) determining whether the patients are suffering from an adenocarcinoma of the prostate or from a benign pathology of the prostate by comparing the value of the ratio determined in step iii) with a predetermined threshold value, chosen according to the type of ratio used and representative of the detection limit of each pathology.

The present invention relates to the diagnosis of adenocarcinomas of the prostate. In particular, the invention relates to a method for diagnosing a benign pathology of the prostate or an adenocarcinoma of the prostate in a patient suspected of suffering from such pathologies, by using binding partners capable of recognizing prostate specific antigen (or PSA) specifically in its activatable free form.

PSA is produced by the glandular epithelium of the human prostate, probably in an inactive zymogen form (Lundwall et al. FEBS Lett. 1987), and is secreted into the seminal fluid in its active form (Lilja, J. Clin Invest 1985). The biological activity of PSA in the seminal fluid is related to its limited proteolytic fragmentation of the predominant proteins secreted by the seminal vesicles (Lilja, J. Clin Invest 1985; Lilja et al. J. Clin Invest 1987; Mc Gee et al. Biol. reprod. 1988).

PSA is the main marker for prostate cancer, which will effect, during his lifetime, one man in six in the West. This protease of the kallikrein family, mainly secreted by the prostatic epithelium, is found at a concentration of 0.5 to 5 mg/ml in the seminal fluid and at a concentration of one million times lower in the serum of a patient. Thus, PSA is normally found at a concentration of less than 2.5 ng/ml in the serum. However, this concentration increases in principle notably when there is a prostate cancer and when there are benign alterations such as benign prostate hypertrophy (BPH) or acute prostatitis.

The protein sequence of PSA has been determined. It is a glycoprotein containing 237 amino acids (“Molecular cloning of human prostate specific antigen cDNA”. Lundwall A., Lilja H., 1987. FEBS Lett 214: 317-322).

A method of diagnosis consisting in measuring the concentration of serum PSA and in comparing it to a threshold value, which is 4 ng/ml, has been proposed. However, it has been noted that this method of diagnosis results in three patients out of four being wrongly suspected, which is prejudicial. In addition, this method does not make it possible to diagnose 30 to 45% of the cases of cancer confined to the gland, which nevertheless constitutes a potentially curable early stage of the disease for which the diagnosis would thus be particularly desirable. The relatively unsatisfactory nature of this method is, moreover, demonstrated by the study reported in the article “Prostate Cancer Detection in Men With Serum PSA Concentrations of 2.6 to 4.0 ng/ml and Benign Prostate Examination”, Catalona et al., JAMA, 14 Mai 1997-Vol. 277, No. 18, which shows that the threshold value must be less than 4 ng/ml for early screening of a prostate cancer.

Moreover, it has been shown that, in the serum, PSA associates with protease inhibitors, such as α-1-antichymotrypsin (ACT) and 1′α-2-macroglobulin (A2M). These associations bring about the inactivation of the chymotryptic activity of PSA, as was demonstrated in the article “Enzymatic activity of prostate specific antigen and its reactions with ultracellular serine proteinase inhibitors” A. Christensson, C. B. Laurell, H. Lilja, 1990 Eur. J. Biochem 194: 755-763. This has also made it possible to demonstrate that the PSA present in the serum is either in a free form, i.e. nonassociated form, or in a complexed form, i.e. associated form. The use of the free PSA to total of the PSA ratio has therefore been proposed in order to improve the specificity of the diagnosis.

Thus, patent application WO 97/12245 describes a method for diagnosing an adenocarcinoma of the prostate without biopsy. This method consists in measuring, in the serum or in the blood of patients, the total amount of PSA. If this value is between 2.5 and 20 ng/ml, the concentration of free PSA is also measured. The free PSA to total PSA ratio is then calculated. If this ratio is less than 7%, the diagnosis is toward an adenocarcinoma of the prostate.

However, the use of a 7% threshold for the diagnosis of prostate cancer is disputed by many authors, as shown by the publication by Lein et al. “Relation of free PSA/total PSA in serum for differentiating between patients with prostatic cancer and benign prostate hyperplasia: which cutoff should be used?”. In this document published in the review Cancer Investigation, 16(1), 45-49, 1998, it was demonstrated that it is difficult, by means of this ratio, to systematically differentiate between a prostate cancer and a BPH.

For these reasons, the applicant focused, in the patent application WO 00/02052, on the presence, in the serum, of cleaved forms of PSA among the free forms of PSA. The molecular forms of serum PSA in patients suffering from cancer or BPH have been mapped by two-dimensional electrophoresis, associated with chemoluminescence detection, in order to observe all the forms of PSA, i.e. the free forms, including the cleaved forms, and the complexed forms.

The electrophoretic profiles of sera from patients suffering from adenocarcinoma of the prostate are relatively homogenous, exhibiting essentially the noncleaved free forms of PSA, while those from individuals suffering from BPH can contain a relatively high proportion of cleaved free forms and slightly more basic spots without cleaved form. The increase in the serum free PSA/serum total PSA ratio observed in patients suffering from BPH is thus thought to be essentially linked to the existence of cleaved free PSA, which could be enzymatically inactive and thus incapable of binding to ACT, and also to the presence of a free PSA form slightly more basic than the active free PSA form, which might correspond to the inactive, zymogen form of PSA.

As a function of these observations, the applicant has described methods for diagnosing adenocarcinoma of the prostate comprising the quantification, after separation by two-dimensional electrophoresis, of the cleaved and/or noncleaved free PSA and the use of these values in order to establish a diagnosis.

However, even though these methods have made if possible to improve the diagnosis, they require the use of two-dimensional electrophoresis. They are consequently quite expensive and require a great deal of handling time.

In order to avoid this handling, patent application WO 01/77180 has described novel antibodies directed specifically against inactive free PSA, and the use thereof in a method for diagnosing benign pathology of the prostate or adenocarcinoma of the prostate.

The applicant has now discovered that there exists, in the biological samples from patients, among the free forms of PSA, a new form of PSA which is not the zymogen form (ProPSA) but is also activatable, i.e. which potentially has the ability to react with protease inhibitors, and that the use of the detection of such an activatable free form of PSA allows a differential diagnosis between individuals suffering from benign pathologies of the prostate and individuals suffering from adenocarcinoma of the prostate, with an excellent specificity and sensitivity.

Thus, a subject of the present invention is a method for the in vitro diagnosis of a benign pathology of the prostate or of an adenocarcinoma of the prostate, characterized in that it comprises the step consisting of detection, in a biological sample from a patient suspected of suffering from a benign pathology of the prostate or from an adenocarcinoma of the prostate, of the activatable free form of PSA.

The various forms of PSA, present in biological fluids such as the seminal fluid or the serum of a patient, have often had various names depending on the authors. They are all in agreement in considering that total PSA is in two forms, namely the free form (free PSA) and the form complexed with protease inhibitors such as ACT (complexed PSA). Free PSA can also be named according to its size: when it is produced in the form of ProPSA, it possesses additional amino acids (7 or 5 or 4 or 2). Reference is then made to zymogen PSA or to ProPSA-7, ProPSA-5, etc. This proPSA, after maturation and therefore loss of additional amino acids, can be either in intact form, or in partial form. In the latter case, reference is made to truncated or cleaved PSA. Free PSA in intact form can also be denatured (glycosylated or deglycosylated); reference is then made to a denatured form. Thus, the free PSA forms include zymogen PSA, intact PSA, which may or may not be denatured, and cleaved PSA.

Reference was also made, for the various free or complexed forms of PSA, to active forms and to inactive forms. The term “active form” is intended to mean the forms which are capable of binding to protease inhibitors, such as ACT. The term “inactive form” is intended to mean the forms incapable of such a binding capacity. It is known that complexed PSA is inactive since it has already bound to an inhibitor. Similarly, the cleaved free PSA and denatured free PSA are inactive since they no longer have the ability to bind to inhibitors. Finally, the zymogen forms of PSA are inactive. The other free forms of PSA are therefore active. Although these active forms are present in the seminal fluid or in supernatants of the LnCaP cell line, it is commonly accepted that these active forms do not exist in the serum because they are complexed with protease inhibitors.

The applicant has now demonstrated, against all expectations, that among these free forms, there exists, in the biological samples of patients, an activatable free form, i.e. a form which still has the ability to bind to protease inhibitors in the serum, it being possible for this binding to be carried out after activation of said activatable free form. It has also demonstrated that the use of the assaying of such an activatable free form of PSA makes it possible to diagnose patients suffering from adenocarcinoma of the prostate or from a BPH.

Without wishing to be bound by any theory, the applicant thinks that this activatable free form of PSA would, after activation, be equivalent to the form of PSA which complexes with ACT. This activatable free form would be a “closed” form of PSA, imprisoning the ACT-binding site, its activation making it possible to convert the “closed” form to the “open” form and thus to free the binding site.

The method of diagnosing of the invention can be carried out either using a binding partner capable of binding specifically to activatable free PSA, or a binding partner capable of binding to activatable free PSA in a nonspecific manner.

Thus, according to a first embodiment, the invention relates to a method for the in vitro diagnosis of a benign pathology of the prostate or of an adenocarcinoma of the prostate, characterized in that it comprises the steps consisting in:

i) bringing a binding partner capable of binding specifically to activatable free PSA into contact with a biological sample from a patient suspected of suffering from a benign pathology of the prostate or of an adenocarcinoma of the prostate,

ii) demonstrating the capture of the activatable free form of PSA by said binding partner,

iii) calculating the ratio of the amount of activatable free form of PSA detected in step ii) to the amount of a form of PSA other than the activatable free form, present in a sample of the same nature taken from the same individual, and

iv) determining whether the patients are suffering from an adenocarcinoma of the prostate or from a benign pathology of the prostate by comparing the value of the ratio determined in step iii) with a predetermined threshold value, chosen according to the type of ratio used and representative of the detection limit of each pathology.

The applicant has discovered that, unexpectedly, among the binding partners suitable for the purposes of the invention, those which recognize the epitope mimicked by the sequence SEQ ID No. 1 (DTPYPWGWLLDEGYD) are capable effectively of binding specifically in the serum to this activatable free form of PSA, without binding either to denatured or cleaved free PSA or to ProPSA, and that the use of this specific property makes it possible to obtain a method for diagnosing prostate-related pathologies which is very sensitive and very specific.

The biological samples in which the method of the invention is carried out are any biological sample liable to contain PSA. By way of example of such samples, mention may be made of seminal fluid, blood, serum, plasma and urine, serum and plasma being particularly preferred.

The binding partners capable of binding specifically to activatable free PSA which are suitable for the purposes of the invention comprise, for example, antibodies, antibody fragments and mimotopes, and also any partner known to those skilled in the art to have this capacity.

The term “antibody fragments” is intended to mean, in general in the present application, any antibody fragment having conserved the specificity of the antibody of origin, in the present case, the ability to bind specifically to activatable free PSA, and in particular fragments of Fab and F(ab′)₂ type. In the present application, the word “antibody” subsequently also denotes antibody fragments when the sense permits.

The antibodies which are useful for the purposes of the invention comprise in particular purified polyclonal antibodies and monoclonal antibodies.

The preparation of polyclonal antibodies and of monoclonal antibodies is widely known to those skilled in the art and the principle of this preparation is recalled hereinafter.

The polyclonal antibodies can be obtained by immunization of an animal with at least one target antigen of interest, followed by recovery of the desired antibodies in purified form, by taking a sample of the serum of said animal, and separating said antibodies from the other constituents of the serum, in particular by affinity chromatography on a column to which is attached an antigen specifically recognized by the antibodies, in particular a target antigen of interest.

The monoclonal antibodies can be obtained by the hybridoma technique, the general principle of which is recalled hereinafter.

Firstly, an animal, generally a mouse (or cells in culture in the case of in vitro immunizations), is immunized with a target antigen of interest, the B lymphocytes for which are then capable of producing antibodies against said antigen. These antibody-producing lymphocytes are subsequently fused with “immortal” myoma cells (murine in the example) so as to give hybridomas. Using the heterogeneous mixture of the cells thus obtained, a selection of the cells capable of producing a specific antibody and of multiplying indefinitely is then carried out. Each hybridoma is multiplied in the form of a clone, each resulting in the production of a monoclonal antibody, the recognition properties of which with respect to the tumor antigen of interest may be tested, for example, by ELISA, by one- or two-dimensional immunoblotting, by immunofluorescence, or using a biosensor. The monoclonal antibodies thus selected are subsequently purified, in particular according to the affinity chromatography technique described above.

The term “mimotopes” is intended to mean any synthetic or recombinant peptide capable of mimicking a conformation which interacts specifically with said epitope.

According to a preferred embodiment, the binding partner capable of binding specifically to activatable free PSA satisfies at least one of the following conditions:
it is capable of recognizing the epitope mimicked by the sequence SEQ ID No. 1;
it is an antibody or an antibody fragment.

The sequence SEQ ID No. 1 recognized by the binding partners suitable for the purposes of the invention mimics a conformational epitope of PSA.

An example of an antibody capable of recognizing the epitope mimicked by the sequence SEQ ID No. 1 suitable for the purposes of the invention is the antibody 5D3D11, as described in Michel S., et al., 1999, Clinical Chemistry, 45(5): 638-650. The use of this particular antibody in the method of the invention is unexpected in the sense that this article indicates that this antibody is capable of inhibiting the enzymatic activity of PSA and thus the binding of ACT; in other words, that it is capable only of binding to an “open” active free form of PSA. In the present case, the applicant has also shown that it is capable of binding to the activatable free form, i.e. “closed” form.

The conjugates consisting of the binding partner capable of binding specifically to activatable free PSA and of the activatable free form of PSA are novel and also constitute a subject of the invention.

According to one embodiment, said binding partner capable of binding specifically to activatable free PSA, of the conjugate of the invention, is a binding partner capable of recognizing the epitope mimicked by the sequence SEQ ID No. 1.

The second step of the method of diagnosis of the invention consists in demonstrating the capture of said activatable free form of PSA by said binding partner capable of binding specifically to activatable free PSA.

This step can be carried out directly by detecting the binding between said binding partner and said activatable free form of PSA, or else after elution of said activatable free form of PSA immunocaptured by said binding partner. The activatable free form of PSA immunocaptured by said binding partner, and then eluted, will subsequently be referred to as immunopurified activatable free PSA.

The elution of the activatable free form of PSA immunocaptured by said binding partner capable of binding specifically to activatable free PSA can be carried out by any method of elution known to those skilled in the art, such as a pH shock. An acidic shock is preferably used, for example using a 0.1M glycine buffer, pH 2.8.

The detection of the capture of said activatable free form of PSA, whether or not it is immunopurified, can be carried out by any means of detection known in the immunoassay field, such as direct detection and indirect detection.

In the case of indirect detection, i.e. by means of a detection partner, a detection partner capable of binding to the activatable free form of PSA is used. This detection partner binds to an epitope different from that used by said binding partner used in step i) when the activatable free PSA is not immunopurified. By way of a detection partner suitable for this purpose, mention may be made of antibodies such as the anti-total PSA antibodies, which constitutes an embodiment of the invention.

Examples of such anti-PSA antibodies capable of recognizing an epitope different from that used by said binding partner used in step i) are described in the article by Michel S., et al (1999) above.

These detection partners can be labeled beforehand.

The term “labeling” is intended to mean the attachment of a label capable of directly or indirectly generating a detectable signal. A nonlimiting list of these labels consists of:
enzymes which produce a signal which is detectable, for example by colorimetry, fluorescence or luminescence, such as horseradish peroxidase, alkaline phosphatase, acetylcholinesterase, β-galactosidase or glucose-6-phosphate dehydrogenase,
chromophores such as luminescent or dye compounds,
radioactive molecules such as ³²P, ³⁵S, or ¹²⁵I,
fluorescent molecules such as fluorescein, rhodomine, alexa or phycocyanins, and
particles such as gold particles, magnetic latex particles, or liposomes.

Indirect labeling systems can also be used, such as, for example, by means of another ligand/anti-ligand pair. The ligand/anti-ligand pairs are well known to those skilled in the art, and mention may, for example, be made of the following pairs: biotin/streptavidin, hapten/antibody, antigen/antibody, peptide/antibody, sugar/lectin, and polynucleotide/sequence complementary to the polynucleotide. In this case, it is the ligand which is bound to the binding partner. The anti-ligand can be directly detectable by means of the labels described in the previous paragraph or can itself be detectable by means of a ligand/anti-ligand.

These indirect systems can result, under certain conditions, in an amplification of the signal. This signal amplification technique is well known to those skilled in the art, and reference may be made to the prior patent applications FR 98/10084 or WO 95/08000 of the applicant or to the article J. Histochem. Cytochem., (1997), 45: 481-491.

The direct detection of the capture of the activatable free form by said binding partner, i.e. without the means of a detection partner, can be carried out, for example, by plasma resonance or by cyclic voltametry on an electrode bearing a conducting polymer.

The direct detection can also be carried out using the specific property of enzymatic activity of the active forms of PSA. In this case, it will be advisable to carry out the activation of the activatable free form of PSA, where appropriate after immunopurification thereof. Indeed, the activation of this activatable free form makes it possible to release the site for binding to the enzymatic substrate, which can then be reacted with an enzymatic substrate. Thus, the detection is carried out by determination of the enzymatic activity of the immunopurified and activated free form of PSA, which constitutes a specific embodiment of the invention.

Examples of enzymatic substrates which are suitable for the purposes of the invention comprise all the substrates which reveal chymotryptic-type protease activity and which are widely known to those skilled in the art. Such substrates are available, for example, from Enzyme System Products. They are composed of a peptide sequence which is recognized and cleaved by PSA, this sequence being coupled to a chromophore or fluorophore group.

The activation of the activatable free form of PSA can be carried out by at least one of the following methods:

bringing said activatable form into contact with a medium having a high salt concentration, of at least 0.15M, preferably of at least 1M, more preferably of at most 2M,

binding the immunopurified activatable free PSA to an antibody capable of increasing the enzymatic activity of PSA,

it being possible for these two methods to be carried out separately, simultaneously or successively, in any order.

By way of a medium having a strong salt concentration, mention may be made of media containing 1.5M NaCl and by way of an antibody capable of increasing the enzymatic activity of PSA, mentioned may be made of the antibody 8G8F5 (bioMérieux, France).

Due to the specific properties of these antibodies capable of increasing the enzymatic activity of PSA, their use in the method of the invention makes it possible to improve the sensitivity of said method.

Thus, according to one embodiment, the method of the invention is characterized in that it uses, in addition to the binding partner capable of binding specifically to activatable free PSA, an antibody capable of increasing the enzymatic activity of PSA, and in particular the antibody 8G8F5.

This antibody capable of increasing the enzymatic activity of PSA can be used as a capture partner in an ELISA assay when the binding partner capable of binding specifically to activatable free PSA is used to immunopurify the activatable free PSA. It can also be used as a detection partner, in particular when the binding partner capable of binding specifically to activatable free PSA is used as a capture partner.

The conjugates consisting of the binding partner capable of binding specifically to activatable free PSA, such as a binding partner capable of recognizing the epitope mimicked by the sequence SEQ ID No. 1, and of the activatable free form of PSA, which is activated, are also novel and constitute a specific embodiment of the invention.

In the method of the invention, the binding partners capable of binding specifically to activatable free PSA, which binds specifically to activatable free PSA, can be used as they are or else in particular in a form attached to a solid support and/or linked to a label.

The attachment of these binding partners to a solid support is well known. The support can be made of any biological or synthetic solid material which has adsorbent properties or is capable of attaching a coupling agent. Materials are known and described in the literature. Among the solid materials capable of attaching these binding partners by adsorption, mention will, for example, be made of polystyrene, polypropylene, latexes, etc. Among the materials which make it possible to attach these binding partners by covalence using a coupling agent, mention may in particular be made of dextran, cellulose, etc. The support can, for example, be in the form of discs, of tubes, of beads, of ticks or of plates, in particular of microtitration plates.

The binding of the binding partners to a label will also make it possible to obtain a direct detection, as described above. This label is as described above.

When the binding partner is not linked to a support or when the activatable free PSA is immunopurified, the method of the invention can also use a capture partner. In the first case, this capture partner is capable of binding to an epitope different from that recognized by the binding partner capable of binding specifically to activatable free PSA.

Examples of a capture partner comprise the anti-PSA antibodies as described above.

Insofar as use is made, in the method of the invention, of both a capture partner and a detection partner for assaying activatable free PSA, these partners bind to different epitopes, themselves being different from the epitope recognized by the binding partner capable of binding specifically to activatable free PSA when the activatable free PSA is not immunopurified.

The methods using the various partners of the activatable form of PSA for demonstrating the capture of the activatable free form of PSA by said binding partner are widely known to those skilled in the art. By way of example, mention may be made of sandwich methods such as the ELISA method, and competition assay methods.

Step iii) of the method of the invention consists in calculating the ratio of the amount of activatable free form of PSA detected in step ii) to the amount of a form of PSA other than said activatable form of the invention, present in a sample of the same nature taken from the same individual.

The expression “sample of the same nature taken from the same individual” is intended to mean either two fractions of the same specimen, or two samples derived from two different specimens but which must be of the same nature, for example serum samples.

The forms other than the activatable free form of PSA are in particular complexed PSA, total PSA, total free PSA, zymogen free PSA, denatured free PSA, cleaved free PSA, and combinations thereof, i.e. all the active or inactive, free or complexed forms of PSA.

The assaying of each of these various forms, in particular using specific antibodies, is known.

The complexed PSA is assayed, for example, using antibodies described in patent application WO 98/22509.

The total PSA is assayed, for example, using antibodies described by H. Nagasaki et al. (1999), Clin. Chem. 45: 4486-496.

Other antibodies capable of binding to various forms of free PSA, including total free PSA, are sold by Chugai (Japan), BiosPacific (Emeryville, Calif.) and Euromedex US Biological (Swampscott, Mass.).

The quantification is carried out in a known manner using the methods for demonstrating the detection of immunoreactions as described above (sandwich, etc.), for example by determining the amount of label when labeling is used.

Of course, when the amounts measured are intended to be compared, these values should be comparable. In other words, the values measured should be related to the same optional dilution or concentration of the sample and also to the same volume.

The various ratios which can be calculated in step iii) of the method of the invention can be chosen from the following:

amount of activatable free PSA/amount of total PSA,

amount of activatable free PSA/amount of total free PSA,

amount of activatable free PSA/amount of complexed PSA,

amount of activatable free PSA/amount of cleaved free PSA,

amount of activatable free PSA/amount of zymogen free PSA,

amount of activatable free PSA/amount of denatured free PSA,

amount of activatable free PSA/amount of (denatured free PSA+zymogen free PSA),

amount of activatable free PSA/amount of (denatured free PSA+cleaved free PSA),

amount of activatable free PSA/amount of inactive free PSA (zymogen, denatured and cleaved)

the inverses of these ratios, or

combinations of these ratios,

the preferred ratios relating to the amount of activatable free PSA to the amount of (denatured free PSA+cleaved free PSA) or the amount of activatable free PSA to the amount of inactive free PSA.

According to a specific embodiment of the invention, the form of PSA other than the activatable free form used to calculate the ratio in step (iii) of the method of the invention is the denatured free PSA form+cleaved free PSA form or the inactive free PSA form.

The implementation of this calculation is obviously carried out as follows:

the amount of activatable free PSA in a biological sample taken from an individual is evaluated using the binding partner capable of binding specifically to activatable free PSA, as described above,

one of the amounts chosen from the amount of total PSA, the amount of total free PSA, the amount of complexed PSA, the amount of zymogen free PSA, the amount of cleaved free PSA, the amount of denatured free PSA, the amount of denatured free PSA+cleaved free PSA, the amount of denatured free PSA+zymogen free PSA and the amount of inactive free PSA, or combinations thereof, is evaluated on a sample of the same nature taken from the same individual, and

said ratio or said inverse ratio, or said combination of ratios, is determined.

Step iv) of the method of the invention consists in determining whether the patients are suffering from an adenocarcinoma of the prostate or of a benign pathology of the prostate by comparing the value of the ratio determined in step iii) to a predetermined threshold value, chosen according to the type of ratio used and representative of the detection limit of each pathology.

It is known that, in general, the results of immunoassays depend to a large extent on the characteristics of specificity and affinity of the antibodies used, and that these characteristics influence the values measured with these antibodies. One therefore imagines that it is not possible to give precise threshold values and that threshold values suitable for each antibody used can be determined in each case by means of simple routine experiments.

It should be clearly understood that the term “threshold value” referred to here is either a discrete value, or a range of values corresponding to a zone of indetermination. Of course, when the value measured is included in the indetermination range, or is very close to the threshold value in the case of a discrete value, it is not possible to reach a definitive conclusion and additional investigations must be carried out.

Of course, when a threshold value has been determined for a given type of ratio, it is possible to deduce therefrom threshold values corresponding to other types of ratios.

When an activatable free PSA/(denatured free PSA+cleaved free PSA) ratio or an activatable free PSA/inactive free PSA ratio is considered, an adenocarcinoma of the prostate will de diagnosed for patients having a ratio above the threshold value obtained using said ratio, and a BPH or other noncancerous prostate pathology (“normal patients”) will be diagnosed for patients having a ratio below this threshold value.

In general, when the amount of activatable free PSA is the numerator of the ratio calculated, an adenocarcinoma of the prostate will be diagnosed for patients having a ratio above the threshold value obtained using said ratio, and a BPH or other noncancerous prostate pathology (“normal patients”) will be diagnosed for patients having a ratio below this threshold value. On the other hand, when the amount of activatable free PSA is the denominator of the ratio calculated, an adenocarcinoma of the prostate will be diagnosed for patients having a ratio below the threshold value obtained using said ratio, and a BPH or other noncancerous prostate pathology (“normal patients”) will be diagnosed for patients having a ratio above this threshold value.

In order to implement the method of the invention, a subject of the invention is also a diagnostic kit for diagnosing an adenocarcinoma of the prostate or a benign pathology of the prostate, said kit comprising:

a binding partner capable of binding specifically to activatable free PSA, preferably a binding partner capable of recognizing the epitope mimicked by the sequence SEQ ID No. 1, more preferably an antibody or an antibody fragment, and

means for assaying the forms of PSA other than the activatable free form, preferably antibodies or antibody fragments.

Such means which can be used in said kit are as described above for assaying the form of PSA that it is desired to quantify.

The method of the invention can also be implemented using a binding partner capable of binding to activatable free PSA in a nonspecific manner.

Such partners may be partners capable of binding with total PSA, including in particular activatable free PSA. By way of example of such partners, mention may be made of antibodies, antibody fragments and mimotopes, and also any other partner known to those skilled in the art to have this capacity, as described above, and in particular the antibody 11E5C6 (Michel et al., 1999, above), the conformational epitope of which involves the C-terminal region of PSA (Michel S et al., 2001, J. Mol. Recognit., 14: 406-413).

This binding partner capable of binding to activatable free PSA in a nonspecific manner can be used in the method of the invention as described above with the binding partner capable of binding specifically to activatable free PSA, i.e. as a capture partner or else for immunopurifying the free PSA.

However, in this specific embodiment, the detection of the activatable free form of PSA must be carried out using the specific property of enzymatic activity of the active forms of PSA. In this case, it is advisable to carry out the activation of the activatable free form of PSA. This is because the activation of this activatable free form makes it possible to free the site for binding to the enzymatic substrate, which may subsequently be reacted with an enzymatic substrate as defined above. Thus, the detection is carried out by determining the enzymatic activity of the free form of PSA which has been activated.

Thus, according to this embodiment, the invention relates to a method for the diagnosis of a benign pathology of the prostate or of an adenocarcinoma of the prostate according to claim 1, characterized in that it comprises the steps consisting in:

i) bringing a binding partner capable of binding to activatable free PSA in a nonspecific manner into contact with a biological sample from a patient suspected of suffering from a benign pathology of the prostate or from an adenocarcinoma of the prostate,

ii) demonstrating the capture of the activatable free form of PSA by said binding partner by determining the enzymatic activity of the activatable free form of PSA, after activation of the activatable free form of PSA,

iii) calculating the ratio of the amount of activatable free form of PSA detected in step ii) to the amount of a form of PSA other than the activatable free form, present in a sample of the same nature taken from the same individual, and

iv) determining whether the patients are suffering from an adenocarcinoma of the prostate or from a benign pathology of the prostate by comparing the value of the ratio determined in step iii) to a predetermined threshold value, chosen according to the type of ratio used and representative of the detection limit of each pathology.

The activation of the activatable free form of PSA can be carried out by means of at least one of the following methods:

bringing said activatable form into contact with a medium having a strong salt concentration, of at least 0.15M, preferably of at least 1M, more preferably of at most 2M, as described above,

binding the activatable free PSA to an antibody capable of increasing the enzymatic activity of PSA, and in particular the antibody 8G8F5,

it being possible for these two methods to be carried out separately, simultaneously or successively, in any order.

Due to the specific properties of these antibodies capable of increasing the enzymatic activity of PSA, their use in the method of the invention makes it possible to improve the sensitivity of said method, which constitutes a preferred embodiment.

Thus, according to this embodiment, the method of the invention is characterized in that it uses, in addition to the binding partner capable of binding to activatable free PSA in a nonspecific manner, an antibody capable of increasing the enzymatic activity of PSA, and in particular the antibody 8G8F5.

This antibody capable of increasing the enzymatic activity of PSA can be used in this respect, in an assay, as a capture partner for activatable free PSA previously immunopurified in a specific or nonspecific manner from biological samples. The free PSA thus activated after capture thereof by the antibody will be detected by means of its enzymatic activity which will be measured by means of hydrolysis kinetics for a fluorescent substrate. The flows of the kinetics will be expressed in fluorescence units. The correspondence in amounts of PSA will be obtained by virtue of a standard curve established with amounts of active PSA.

With the exception of the preceding text, steps i) to iv) of this specific embodiment are as previously described.

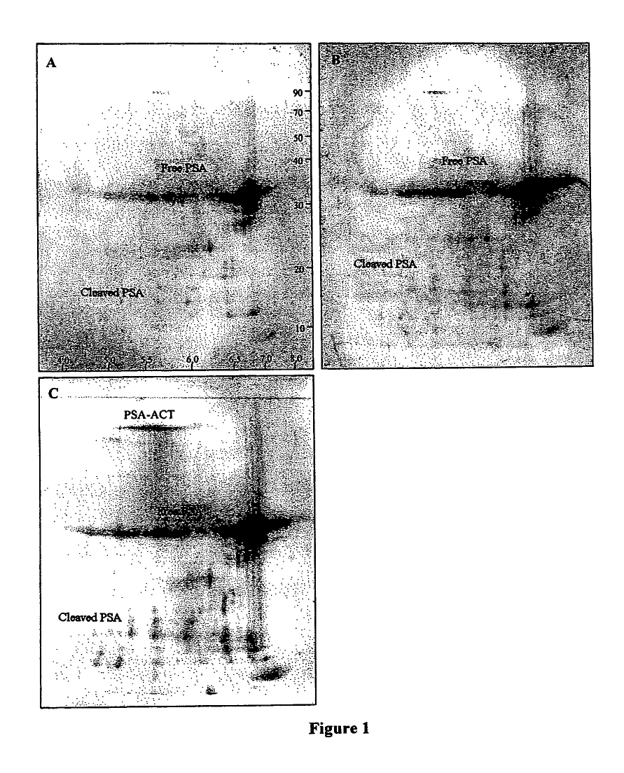

The present invention will be understood more fully from the following examples given only by way of nonlimiting illustration, and also from the attached FIGS. 1 to 6, in which:

FIG. 1 shows the photograph of 3 two-dimensional electrophorese gels, obtained using 0.5 μg of PSA derived from the culture supernatant of LNCaP cells, immunopurified with the antibody 5D3D11 (photograph A), 6C8D8 (photograph B) and 11 E5C6 (photograph C),

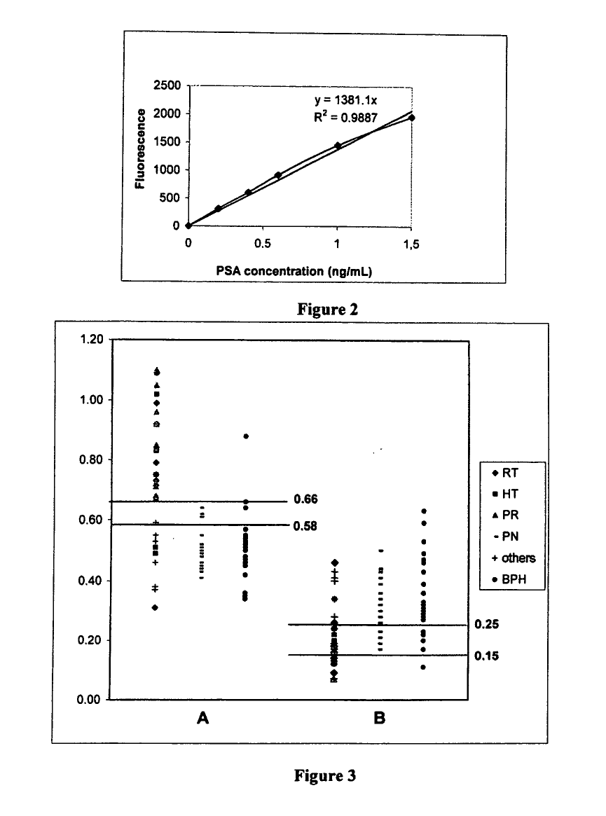

FIG. 2 represents a standardization graph giving the fluorescence as a function of the PSA concentration obtained using seminal PSA previously immunopurified with the antibody 5D3D11 and assayed in terms of total PSA,

FIG. 3 is a graphic representation giving the values of the activatable PSA/(cleaved free PSA+denatured free PSA) ratios obtained with the method of the invention for serum from patients suffering from prostate cancer and treated by radiotherapy (RT), by hormone therapy (HT) or by prostatectomy (PR), from patients suffering from cancer but for whom the treatment is not known (others), from patients suffering from benign hyperplasias (BPH) and from normal patients (PN) (part A of the graph), and also the values of the free PSA/total PSA ratios obtained according to a method of the prior art (part B of the graph) with these same serum,

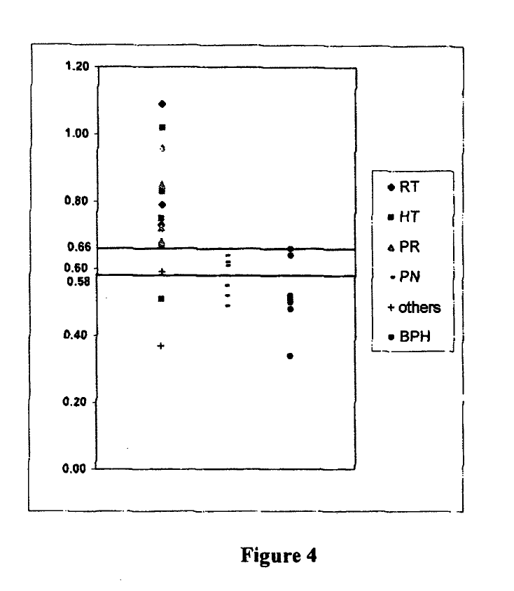

FIG. 4 is a graphic representation giving the values of the activatable PSA/(cleaved free PSA+denatured free PSA) ratios obtained with the method of the invention in the sera having a free PSA/total PSA ratio of between 0.15 and 0.25, the sera being from patients suffering from prostate cancer and treated by radiotherapy (RT), by hormone therapy (HT) or by prostatectomy (PR), from patients suffering from cancer but for whom the treatment is not known (others), from patients suffering from benign hyperplasias (BPH) and from normal patients (PN),

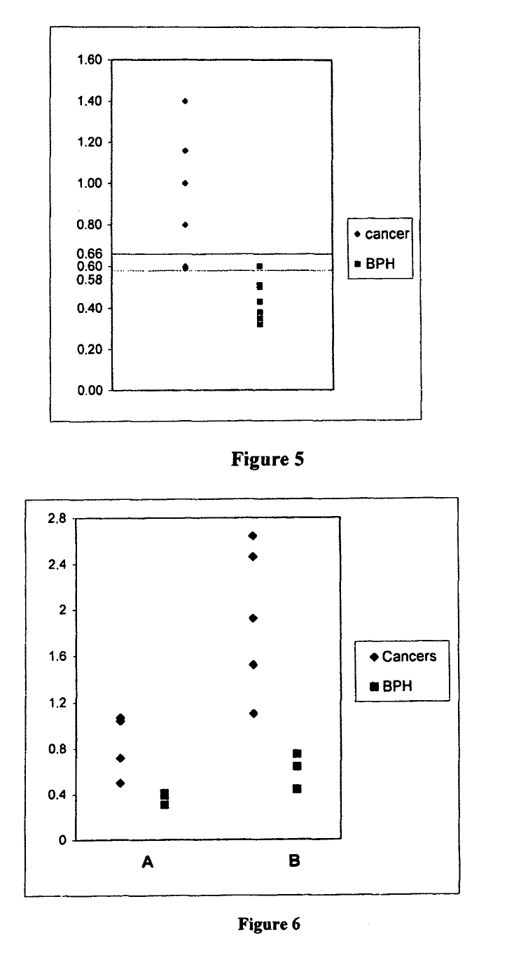

FIG. 5 is a graphic representation giving the values of the activatable PSA/(cleaved free PSA+denatured free PSA) ratios for the sera from patients suffering from prostate cancer and from patients suffering from benign hyperplasia (BPH), for whom the total PSA level is less than 2.5 ng/ml, and

FIG. 6 is a graphic representation giving the values of the activatable PSA/(cleaved free PSA+denatured free PSA) ratios for the sera from patients suffering from prostate cancer and from patients suffering from benign hyperplasias (BPH), obtained with the antibody 5D5A5 or the antibody 11E5C6 as antibodies for detecting activatable PSA.

### EXAMPLE 1

### Detection of the Activatable Free Form of PSA Using LNCaP Supernatants

1.1 Preparation of LNCaP Supernatants

The LNCaP cell line was made to synthesize PSA according to the technique described in the articles “LNCaP Produces both putative zymogen and inactive, free form of prostate specific antigen” E. Corey et al., 1998, Prostate 35:135-143, “Androgen-Sensitive human prostate cancer cell, LNCaP, Produce both N-terminally mature and tuncated prostate specific antigen isoform” A. Herrala et al., 1997, Eur. J. Biochem. 255:329-335, “Production of milligram concentration of free prostate specific antigen (fPSA) from LNCaP cell culture: Difference between fPSA from LNCaP cell and seminal plasma.” J. T. Wu et al., 1998, J. Clin. Lab. Anal. 12: 6-13.

The PSA thus produced by the LNCaP cell line is composed of free PSA. In addition, since the culturing of the line is carried out in the presence of fetal calf serum, which contains ACT, a proportion of PSA-ACT complex is thus also possible.

1.2 Binding of the Free Forms of PSA in the Supernatant by Binding Antibodies

Either the antibody 5D3D11, which is a binding partner which recognizes the epitope mimicked by the sequence SEQ ID No. 1, or the antibody 6D8C8, which is a binding partner which does not recognize this epitope (Michel S., et al., 1999, above), is used.

The binding is carried out as follows:

The 5D3D11 or 6C8D8 antibodies were attached to a CNBr activated sepharose resin support (Pharmacia) according to the conventional protocol provided by the manufacturer, well known to those skilled in the art. The coupling ratio used was 5 mg of antibody per 1 ml of swollen resin. 1 ml of resin attached to 5 mg of antibody was brought into contact with 200 ml of a pool of LNCaP cell supernatants containing approximately 500 μg of total PSA assayed using the Vidas detection device (total PSA kit, bioMérieux, France). After 2 passages over the affinity column, the section containing the unbound PSA was conserved (called F1) and the PSA bound by the antibodies was eluted with a 0.1M glycine buffer, pH 2.8, subsequently neutralized to pH 7.2 with 2M Tris, pH 8. The free forms of PSA were thus obtained, immunopurified either with the antibody 5D3D11 or the antibody 6C8D8.

1.3 Assaying of the Free Forms of PSA in the Supernatant after Immunopurification with the Antibodies 5D3D11 and 6C8D8

1.3.1. Analysis by Two-Dimensional Electrophoresis

The PSA contained in the various fractions derived from the immunopurification was analyzed by two-dimensional electrophoresis and Western Blot.

During the separation in the first dimension, the proteins migrate according to their isoelectric point (pI) by isoelectric focusing (IEF) on a pH gradient immobilized on deposit strips (Immobiline Dry-strip pH 3-10, 18 cm, nonlinear, Pharmacia) under denaturing and reducing conditions. 0.5 μg of PSA was added to 10 μl of a solution containing 10% of SDS and 2.3% of DTT (dithio-1,4-threitol), and the mixture was heated at 96° C. for 5 min. The sample was then made up to 500 μl with the rehydration solution (8.3 M urea, 2 M thiourea, 4% CHAPS (3-[(3-cholamidopropyl)dimethylammonio]-1-propanesulfonate), 100 mM DTT, 2% Servalyt 4-9, 1 mg/ml Orange G), and brought into contact with the strips in a 20 cm-long glass test tube. The whole was then covered with paraffin oil and incubated overnight. The separation was carried out under a voltage increasing in a linear manner from 100 to 3500 V in 8 h, and a focusing step at 6000 V was carried out for 80 to 100 kVh.

The proteins which had focused at their isoelectric point were then separated in a second dimension according to their size by means of an SDS-PAGE electrophoresis, on a large homogenous 12% acrylamide gel at 40 mA per gel, for 5-6 h, according to the conventional protein electrophoresis technique.

The gels obtained after separation according to the second dimension were blotted onto a PVDF membrane (polyvinylidene trifluoride; millipore) in CAPS/methanol buffer (3-[cyclohexilamino]-1-propanesulfonic acid), overnight under a current of 1 A, maintaining the temperature at 15° C. The membranes were saturated overnight at 4° C. in TBS (Tris Buffered Saline, 15 mM Tris, pH 8, 140 mM NaCl) containing 0.05% of Tween 20 and 5% of dehydrated skimmed milk. The anti-PSA antibody 13C9E9, previously identified for detecting all forms of PSA (Charrier J. P., et al., 2001, Electrophoresis, 22, 1861-1866), was diluted to 10 μg/ml in the saturation solution, and was added to the membranes. After incubation at 1 h at 37° C., the membranes were washed three times (5 min) with a saturation solution, and brought into contact with the peroxidase-coupled anti-mouse antibody conjugate (Jackson ImmunoResearch, West Grove, United States of America), diluted to 1/5000th in the saturation solution. They were then incubated at ambient temperature for 1 h, and washed three times in saturation solution. The immunoreactivity was detected using a chemoluminescent substrate (Pierce), by incubating the membranes in Fluor S, and performing an image acquisition (between 1 min and 1 h according to the sample). The images obtained were then processed using the Multi-Analyst software (Biorad).

The photographs of these gels are reproduced in FIG. 1, where photograph A corresponds to the use of the antibody 5D3D11, photograph B corresponds to the use of the antibody 6C8D8 and photograph C corresponds to the antibody 1 E5C6 (Michel S. et al., 1999, above) which is an anti-total PSA antibody used by way of comparison.

This figure shows that, by comparison with the PSA immunopurified with the anti-total PSA antibody 11E5C6, the PSA immunopurified with the antibody 5D3D11 contains very few cleaved or truncated forms. On the other hand, these forms are present in much greater proportion in the PSA immunopurified with the 6C8D8 antibody.

1.3.3 Characterization of the Zymogen and Mature Forms of the Immunopurified PSA by N-Terminal Sequencing of the PSA

5 to 8 μg of cellular PSA immunopurified with the antibody 5D3D11 or 6C8D8 was subjected to SDS-PAGE gel separation under reducing conditions and blotted onto a polyvinylidene difluoride (PVDF) membrane according to the techniques well known to those skilled in the art. The PSA bands were stained with a solution of Ponceau Red S at 0.25% in 3% TCA (trichloroacetic acid), and the band corresponding to the mature PSA (approximately 30 kDa) was cut out and subjected to Edman degradation on a Procise 292A protein sequence (Applied Biosystems).

The results obtained showed that the PSA immunopurified with the antibody 5D3D11 contained only the mature form (IVGG . . . ), whereas the PSA immunopurified with the antibody 6C8D8 contained the mature form and also proPSA(−7) and proPSA(−5).

1.3.4 Measurement of the Enzymatic Activity of the Immunopurified Cellular PSA

The wells of a Nunc Maxisorp black ELISA plate (compatible with fluorescence detection) were coated for 2 hours at 37° C. with 100 μl of a solution of streptavidin at 10 μg/ml in carbonate buffer, pH 9.6, and blotted for 2 hours at 37° C. with TBS (50 mM Tris-HCl, pH 7.5, 0.15 M NaCl) containing 2 mg/ml of BSA (Bovine Serum Albumin). After 3 washes of the wells in TBS-0.05% Tween, 100 μl of biotinylated anti-total PSA antibody diluted to 10 μg/ml in TBS-BSA were introduced. The anti-total PSA antibody can be either the antibody 11E5C6 which does not modify the enzymatic activity of PSA (Michel S. et al., 1999, above), or the antibody 8G8F5 (bioMérieux, France).

After incubation for 2 hours at 37° C., the wells were washed with TBS-0.05% Tween, and the PSA immunopurified with the antibody 5D3D11 or 6C8D8, diluted in TBS-BSA to 2.5 μg/ml, was introduced and incubated overnight at 4° C. The wells were then washed again in TBS-0.05% Tween and incubated for 15 min. either with the TBS-BSA described above (containing 0.15 M of NaCl), or with TBS-BSA in which the NaCl concentration was increased to 1.5 M. The wells were then emptied and brought into contact with 100 μl of fluorescent substrate Mu-HSSKLQ-AFC (Enzyme System Products, subsidiary of ICN) diluted to 300 μM in TBS-BSA containing either 0.15 M, or 1.5 M of NaCl. The enzymatic kinetics were followed at 37° C. for 2 hours (excitation 390 nm, emission 510 nm; 1 measurement/minute) with the Fluoroskan reader (Thermolabs Systems). The enzymatic activity of the PSA corresponds to the slope of the curve obtained.

The results are indicated in Table 1 below:

TABLE 1

| | Capture with | | Capture with | |
|---|---|---|---|---|
| | biotinylated | | biotinylated | |
| | 11E5C6 | | 8G8F5 | |
| | 0.15 M | 1.5 M | 0.15 M | 1.5 M |
| | NaCl | NaCl | NaCl | NaCl |
| LNCaP supernatant | Not | Not | Not | Not |
| | detectable | detectable | detectable | detectable |
| Cellular PSA | 17.1 | 51.8 | 79.9 | 153.1 |
| immunopurified with | | | | |
| 5D3D11 | | | | |
| Cellular PSA | Not | Not | Not | 7.0 |
| immunopurified with | detectable | detectable | detectable | |
| 6C8D8 | | | | |

The enzymatic activity is expressed as fluorescence units ×1000/minute.

This table shows that the PSA immunopurified with the antibody 5D3D11 has a detectable enzymatic activity. This activity is increased in the presence of 1.5 M NaCl. The capture of the PSA with the antibody 8G8F5 also increases the enzymatic activity of the PSA.

The PSA immunopurified with the antibody 6C8D8, for its part, has only a residual activity detectable in the presence of 1.5M NaCl and of the antibody 8G8F5, showing that this immunopurified form of PSA is not activatable.

This example demonstrates that, among the active free forms of PSA in the LNCaP cells, the activatable free form which is detectable according to the method of the invention is found.

### EXAMPLE 2

### Detection of the Activatable Free Form of PSA Using Serum from Patients Having an Adenocarcinoma of the Prostate

The pool of serum used is composed of 30 sera from patients having a prostate cancer. PSA concentration of this pool: total PSA: 71 ng/ml; free PSA: 12 ng/ml, determined using the Vidas device.

2.1. Immunopurification of Serum

The resin described in paragraph 1.2 is used as solid support having attached the antibodies 5D3D11 and 6C8D8. 50 μl of these resins are brought into contact with 1 ml of the pool of serum described above, overnight at 4° C. with agitation. The tubes were then centrifuged, the fraction containing the unbound PSA was conserved, the resin washed 3 times with PBS-0.5% Tween (the washing fractions are combined and conserved), and then the PSA bound by the antibody 5D3D11 or 6C8D8 was eluted for 5 min with 200 μl of 0.1 M glycine, pH 2.2, containing 1 mg/ml of BSA, and neutralized to pH 7.2 with 2 M Tris, pH 8.

2.1. Measurement of the Enzymatic Activity of the PSA Immunopurified from Serum

The wells of a Nunc Maxisorp black ELISA plate (compatible with fluorescence detection) were “coated” for 2 hours at 37° C. with 100 μl of a solution of streptavidin at 10 μg/ml in carbonate buffer, pH 9.6, and blocked for 2 hours at 37° C. with TBS (50 mM Tris-HCl, pH 7.5, 0.15 M NaCl) containing 2 mg/ml of BSA (Bovine Serum Albumin). After 3 washes of the wells in TBS-0.05% Tween, 100 μl of biotinylated anti-total PSA antibody diluted to 10 μg/ml in TBS-BSA were introduced. The anti-total PSA antibody can be:
either the antibody 11 E5C6, which does not modify the enzymatic activity of the PSA,
or the antibody 8G8F5 which increases the enzymatic activity of the captured PSA.

After incubation for 2 hours at 37° C., the wells were washed with TBS-0.05% Tween, and 100 μl of the PSA immunopurified from serum with the antibody 5D3D11 or 6C8D8 were introduced and incubated overnight at 4° C. The wells were then washed again in TBS-0.05% Tween and incubated for 15 min with TBS-2 mg/ml BSA, in which NaCl concentration was increased to 1.5 M. The wells were then emptied and brought into contact with 100 μl of the fluorescent substrate Mu-KGISSQY-AFC (Enzyme System Products, subsidiary if ICN) diluted to 400 μM in TBS-BSA containing 1.5 M of NaCl. the enzymatic kinetics were followed at 37° C. for 2 hours (excitation 390 nm, emission 510 nm; 1 measurement/minute) with the Fluoroskan reader (Thermolabs Systems). The enzymatic activity of the PSA corresponds to the flow of the curve obtained.

The results are indicated in Table 2 below:

TABLE 2

| | 1.5 M NaCl | |
|---|---|---|
| | Biotinylated | Biotinylated |
| | 11E5C6 | 8G8F5 |
| Initial serum pool | 1.0* | 4.9* |
| Serum PSA immunopurified with 5D3D11 | 29.5 | 62.9 |
| Serum PSA immunopurified with 6C8D8 | 4.4 | 11.5 |

The enzymatic activity is expressed in fluorescence units ×1000/minute.
*The activity measured directly from the serum (without immunopurification) is underestimated because the activated PSA complexes immediately with the sera ACT.

This table shows that it is possible to measure an enzymatic activity on PSA immunopurified from serum: the serum therefore contains activatable free PSA.

A significant difference in activity is always clearly noted between the PSA immunopurified with the antibody 5D3D11 and that immunopurified with the antibody 6C8D8, showing that the antibody 5D3D11 specifically recognizes the activatable free PSA.

### EXAMPLE 3

### Use of the Antibody 5D3D11 in a Diagnostic Test for Discriminating Between a BPH and Adenocarcinoma of the Prostate

For the assay, the Vidas device is used, adapted to include the appropriate assay reagents, but used according to the supplier's instructions (bioMérieux, France).

3.1 Assaying of the Activatable PSA in Sera from Patients

The antibody 5D3D11 is used as capture antibody in a sandwich assay. The PSA captured is detected with the anti-total PSA antibody 5D5A5 coupled to alkaline phosphatase. The protocol is also described above.

The standardization of the assay was carried out according to the supplier's recommendations using seminal PSA immunopurified with the antibody 5D3D11 and assayed with respect to total PSA. The detection limit of the assay is 0.05 ng/ml of active PSA/ml. The standardization curve in the concentration range of 0 to 1.5 ng/ml is represented in FIG. 2. This curve, which is close to a straight line, demonstrates that assaying with a binding partner which recognizes the epitope mimicked by the sequence SEQ ID No. 1 makes it possible to detect activatable free PSA with good sensitivity.

3.2 Assaying of (Cleaved Free PSA+Denatured Free PSA) in Sera from Patients

The procedure described above was repeated, except that the anti-free PSA antibody 6C8D8 was used as capture antibody for capturing, on the serum, the inactive free PSA forms, and the anti-total PSA antibody 11E5C6, coupled to alkaline phosphatase, was used as detection antibody, which antibody recognizes, in the present case, the cleaved free and denatured free PSA forms.

3.3 Use of the Activatable PSA/(Cleaved Free PSA+Denatured Free PSA) Ratio for Better Discrimination Between a Prostate Cancer and a BPH

A retrospective study was carried out with 177 sera from patients, containing PSA. These sera correspond to:

89 prostate cancers clearly identified with the total PSA levels, the tumor grades, the Gleason scores and possible information on the treatments (radiotherapy, hormone therapy, prostatectomy or unknown) subsequently carried out,

65 benign hyperplasias,

23 normal prostates (having PSA levels>2.5 ng/ml but negative biopsies).

The activatable free PSA/(denatured free PSA+cleaved free PSA) ratio, obtained using the above assays, was calculated on the sera and these values were produced on the graph in FIG. 3, part A.

As shown in part A of FIG. 3, using a threshold of 0.66 for sera having a total PSA level of between 2.5 and 10 ng/ml, the activatable PSA/(cleaved free PSA+denatured free PSA) ratio makes it possible to characterize 22/33 cancers (67%), 19/19 normal prostates (100%) and 27/28 BPH (96%).

By way of comparison, the free PSA/total PSA ratio is calculated using two Vidas kits (total PSA kit and free PSA kit, bioMérieux, France) according to the supplier's recommendations. The results are reproduced on part B of the graph in FIG. 3. This part B shows that a zone of uncertainty exists for values of 0.15 to 0.25.

3.4 Sensitivity of the Method of the Invention

3.4.1 For the Sera Included in the Zones of Uncertainty of the Method of the Prior Art

The values of the activatable free PSA/(denatured free PSA+cleaved free PSA) ratios obtained according to the method of the invention for the 28 sera of the zone of uncertainty obtained with the method of the prior art were reproduced in FIG. 4.

This figure demonstrates that, for the sera for which the free PSA/total PSA ratio is between 0.15 and 0.25, sera in which the known methods do not make it possible to distinguish between a cancer and a BPH, the activatable PSA/(cleaved free PSA+denatured free PSA) ratio makes it possible to characterize 13/15 cancers (80%), 7/7 BPH (100%) and 6/6 normal prostates (100%).

3.4.2 For the Sera Having a PSA Level Less than 2.5 Ng/Ml

The 20 sera from patients suffering from cancer (11) or from BPH (9) having a PSA level of less than 2.5 ng/ml were selected and the values of the activatable free PSA/(denatured free PSA+cleaved free PSA) ratios are reproduced in FIG. 5.

This figure demonstrates that the discrimination between a prostate cancer and a BPH using the activatable PSA/(denatured free PSA+cleaved free PSA) ratio also remains valid for sera having a PSA level of less than 2.5 ng/ml.

3.5 Modification of the Detection Antibody

The above procedure was repeated with 8 sera, 5 sera from patients suffering from prostate cancer and 3 sera from patients suffering from BPH, except that the antibody 11E5C6 (Michel S., et al., 1999, above) was used as detection antibody.

The results are indicated in FIG. 6, in which the values of the activatable free PSA/(denatured free PSA+cleaved free PSA) ratios using the antibody 5D5A5 as antibody for detecting the activatable PSA are reproduced in part A of the curve, and the values of the activatable free PSA/(denatured free PSA+cleaved free PSA) ratios obtained with the same sera but with the antibody 11E5C6 as detection antibody are reproduced in part B.

This figure demonstrates that a threshold value clearly exists whatever the detection antibody used, and that the threshold value depends on the antibody used in detection.

### EXAMPLE 4

### Detection of the Activatable Free Form of PSA Using a Binding Partner Capable of Binding in a Nonspecific Manner to the Activatable Free PSA in a Biological Sample

1. Samples Used

Nine individual sera having total PSA concentrations ranging from 157 to 3.8 ng/ml and originating from the CHU [University Teaching Hospital] of Liège, or from the hôspital des Armées, Desgenettes [Desgenettes Military Hospital], at Lyon, were used.

2. Immunopurification of PSA Using the Antibody 11E5C6

2.1. Coupling of the Antibody with Beads

10⁸ beads coated beforehand with streptavidin (Dynabeads® M-280 Streptavidin, Dynal), i.e. 150 μl of bead solution, were introduced into tubes and were washed 3 times with 500 μl of 0.1 M phosphate buffer, pH 7.4, 0.15 M NaCl, 0.1% BSA to which 0.5% Tween 20 has been added (buffer D+0.5% T). Each wash was carried out at ambient temperature for 5 min, on a wheel in order for the solution to remain homogeneous.

The 10⁸ beads were coupled with 500 μl of antibody 11E5C6 (bioMérieux, Marcy, France) at 20 μg/ml, i.e. 10 μg of antibodies, in buffer D+0.05% T at ambient temperature, for 30 min on a wheel. After coupling, 5 μl of 10 mM biotin were added to the 500 μl of antibody solution and incubated for 30 min at ambient temperature on the wheel.

In order to remove the excess biotin and antibodies, the beads were then washed 5 times with 500 μl of buffer D+0.5% T on the wheel for 5 min at ambient temperature.

2.2. PSA Binding

1 ml of sample containing the PSA (either dilute seminal PSA used as a control, or serum) was introduced into the tubes containing the 10⁸ beads and was incubated overnight at 4° C.

After this incubation period, the unbound PSA was recovered and assayed in order to determine the residual amount of PSA.

Three washes were then carried out, in order to detach the proteins bound nonspecifically, with 500 μl of buffer D having a Tween 20 concentration of 0.5%.

2.3. Elution

The PSA was eluted for 5 min at ambient temperature with 100 μl of 0.2 M Tris-glycine, pH 2.2, to which 1 mg/ml of BSA had been added. The acidic pH results in cleavage of the PSA-Ab bond. The eluate recovered was neutralized with 11 μl of 0.1 M Tris pH 9.6, in order to avoid degradation of the protein due to the acidity of the solution and preserve its enzymatic activity.

3. Assaying of the Samples with Respect to Free PSA and Total PSA

This assay was carried out using the VIDAS device (bioMérieux, Marcy, France), using the assay kits measuring total PSA (TPSA) and free PSA (FPSA), according to the supplier's recommendations and according to the following protocol: specific antibodies directed respectively against total PSA or free PSA are attached to the tips. For the two assays, the detection antibody is the antibody 11 E5C6 coupled to alkaline phosphatase, which catalyzes the reaction for hydrolysis of the substrate to product, the fluorescence of which is emitted and measured at 450 nm.

Each assay requires 200 μl of sample, which are deposited into the bar and the results are available in 1 h. The TPSA assay has a sensitivity of 0.07 ng/ml of PSA and can detect up to 100 ng/ml of PSA. The FPSA assay has a sensitivity of 0.05 ng/ml and can detect up to 10 ng/ml of PSA.

4. Measurement of the Enzymatic Activity of PSA

4.1 Attachment of the Capture Antibodies

The wells of an ELISA plate were coated with 100 μl of streptavin diluted to 10 μg/ml in carbonate buffer, pH 9.6, and were incubated for 2 h at 37° C. The wells were then saturated with 250 μl of 50 mM Tris buffer+0.15 M NaCl, pH 7.5 (TBS) to which BSA had been added at 2 mg/ml (TBS+BSA) in order to avoid nonspecific attachments and therefore to limit the background noise. After 3 washes in TBS containing 0.05% Tween 20 (TBS+0.05% T), 100 μl of biotinylated antibody 8G8F5 (bioMérieux, Marcy, France) at 10 μg/ml were then added and incubated for 2 h at 37° C. This step was followed by 3 washes in TBS+0.05% T.

4.2. Incubation of PSA

85 μl of PSA diluted in TBS+BSA (seminal PSA or immunopurified sera) were introduced into the wells and incubated overnight at 4° C. The wells were then washed 3 times in TBS+0.05% T, which makes it possible to eliminate the unbound PSA. The enzyme was then incubated for 15 min at 37° C., with 100 μl of the substrate+TBS+BSA to which NaCl had been added at 1.5 M.

4.3. Visualization and Assay

The substrate KGISSQY-AFC was diluted in TBS+BSA to which NaCl had been added at 1.5 M. 100 μl of this dilute substrate were then added to the wells and incubated at 37° C. The fluorescence emitted was measured every minute for 2 h at 37° C., using the Fluoroskan device (ThermoLabsystem).

The enzymatic activity of the enzyme was determined by calculating the slope of the kinetics, and was expressed in fluorescence units×1000/min.

5. Results

The results are indicated in Table 3 below.

TABLE 3

| Characterization of sera | | | | Measurement of the enzymatic activity of the PSA | | | |
|---|---|---|---|---|---|---|---|
| | Total | | | Amount | | | Specific activity |
| | PSA | Free PSA | | of free | Enzymatic activity | | (fluorescence |
| Serum | conc. | conc. | Free PSA/ | PSA/well | (fluorescence | | unit/min. × 1000/ |
| reference | (ng/ml) | (ng/ml) | total PSA ratio | (ng) | unit/min. × 1000) | Error | ng of free PSA) |
| 1 | 152.6 | 10.4 | not applicable | 8.12 | 90 | 0.1 | 11.1 |
| 2 | 44.6 | 5.5 | not applicable | 2.91 | 8.5 | 0.1 | 2.9 |
| 3 | 20.5 | 7.4 | not applicable | 6.28 | 3.6 | 0 | 0.6 |
| 4 | 13.1 | 3.3 | not applicable | 1.97 | 4 | 0.1 | 2.1 |
| 5 | 11 | 2.1 | not applicable | 1.76 | 4 | 0 | 2.4 |
| 6 | 10.5 | 2.4 | 0.23 | 1.32 | 3.8 | 0.1 | 2.9 |
| 7 | 8.6 | 2.6 | 0.30 | 1.6 | 4.5 | 0 | 2.8 |
| 8 | 5 | 2.1 | 0.42 | 1.22 | 1.6 | 0.1 | 1.3 |
| 9 | 3.8 | 0.4 | 0.11 | 0.25 | 1.7 | 0.1 | 6.8 |

Among these sera, serum 1 and serum 9 originate from patients confirmed to be suffering from prostate cancer, which is also confirmed by the method of the invention.

1. A method for the in vitro diagnosis of a benign pathology of the prostate or of an adenocarcinoma of the prostate, comprising:
detecting, in a biological sample from a patient suspected of suffering from a benign pathology of the prostate or from an adenocarcinoma of the prostate, the activatable free form of PSA.

2. The method for the diagnosis of a benign pathology of the prostate or of an adenocarcinoma of the prostate as claimed in claim 1, comprising:
i) bringing a binding partner capable of binding specifically to activatable free PSA into contact with a biological sample from a patient suspected of suffering from a benign pathology of the prostate or of an adenocarcinoma of the prostate,
ii) demonstrating the capture of the activatable free form of PSA by said binding partner,
iii) calculating the ratio of the amount of activatable free form of PSA detected in step ii) to the amount of a form of PSA other than the activatable free form, present in a sample of the same nature taken from the same individual, and
iv) determining whether the patients are suffering from an adenocarcinoma of the prostate or from a benign pathology of the prostate by comparing the value of the ratio determined in step iii) with a predetermined threshold value, chosen according to the type of ratio used and representative of the detection limit of each pathology.

3. The method for the diagnosis of a benign pathology of the prostate or of an adenocarcinoma of the prostate as claimed in claim 2, wherein said binding partner used in step i) is capable of recognizing the epitope mimicked by the sequence SEQ ID No. 1

4. The method for the diagnosis of a benign pathology of the prostate or of an adenocarcinoma of the prostate as claimed in claim 2, wherein said binding partner used in step i) is an antibody or an antibody fragment.

5. The method for the diagnosis of a benign pathology of the prostate or of an adenocarcinoma of the prostate as claimed in claim 2, wherein the demonstration of the capture of the activatable free form of PSA by said binding partner is carried out by indirect detection by means of a detection partner, preferably using an anti-total PSA antibody.

6. The method for the diagnosis of a benign pathology of the prostate or of an adenocarcinoma of the prostate as claimed in claim 2, wherein the demonstration of the capture of the activatable free form of PSA by said binding partner is carried out by determining the enzymatic activity of the immunopurified and activated, activatable free form of PSA.

7. The method for the diagnosis of a benign pathology of the prostate or of an adenocarcinoma of the prostate as claimed in claim 2, wherein it uses, in addition to the binding partner capable of binding specifically to activatable free PSA, an antibody capable of increasing the enzymatic activity of PSA.

8. The method for the diagnosis of a benign pathology of the prostate or of an adenocarcinoma of the prostate as claimed in claim 2, wherein the form of PSA other than the activatable free form used for calculating the ratio is the inactive free form of PSA or the cleaved and denatured free forms of PSA.

9. A diagnostic kit for diagnosing an adenocarcinoma of the prostate or a benign pathology of the prostate, comprising:
a binding partner capable of binding specifically to activatable free PSA, and
means for assaying the forms of PSA other than the activatable free form.

10. The diagnostic kit as claimed in claim 9, wherein said binding partner is capable of recognizing the epitope mimicked by the sequence SEQ ID No. 1, preferably an antibody or an antibody fragment.

11. The diagnostic kit as claimed in claim 9, wherein said means are antibodies or antibody fragments.

12. A conjugate consisting of a binding partner capable of binding specifically to activatable free PSA and of the activatable free form of PSA.

13. The conjugate as claimed in claim 12, wherein said binding partner is capable of recognizing the epitope mimicked by the sequence SEQ ID No. 1.

14. The conjugate as claimed in claim 12, wherein the activatable free form of PSA is activated.

15. The method for the diagnosis of a benign pathology of the prostate or of an adenocarcinoma of the prostate as claimed in claim 1, comprising:
i) bringing a binding partner capable of binding to activatable free PSA in a nonspecific manner into contact with a biological sample from a patient suspected of suffering from a benign pathology of the prostate or from an adenocarcinoma of the prostate,
ii) demonstrating the capture of the activatable free form of PSA by said binding partner by determining the enzymatic activity of the activatable free form of PSA, after activation of the activatable free form of PSA,
iii) calculating the ratio of the amount of activatable free form of PSA detected in step ii) to the amount of a form of PSA other than the activatable free form, present in a sample of the same nature taken from the same individual, and
iv) determining whether the patients are suffering from an adenocarcinoma of the prostate or from a benign pathology of the prostate by comparing the value of the ratio determined in step iii) to a predetermined threshold value, chosen according to the type of ratio used and representative of the detection limit of each pathology.

16. The method for the diagnosis of a benign pathology of the prostate or of an adenocarcinoma of the prostate as claimed in claim 15, wherein the activation of the activatable free form of PSA is carried out using an antibody capable of increasing the enzymatic activity of PSA.


SEQUENCE LISTING (1 sequence)

SEQ ID NO: 1 - protein, 15 aa, Artificial Sequence
  epitope
DTPYPWGWLLDEGYD